(19) United States
(12) Patent Application Publication
(10) Pub. No.: US 2006/0240393 A1
(43) Pub. Date: Oct. 26, 2006

(54) System, method, and computer program product for an automated neuropsychological test

(76) Inventors: Dennis L. Reeves, Springfield, MO (US); Kathryn P. Winter, Pensacola Beach, FL (US)

Correspondence Address: OFFICE OF THE STAFF JUDGE ADVOCATE;U.S. ARMY MEDICAL RESEARCH AND MATERIEL COMMAND, ATTN: MCMR-JA (MS. ELIZABETH ARWINE), 504 SCOTT STREET, FORT DETRICK, MD, 21702-5012, US

(21) Appl. No.: 11/305,332

(22) Filed: Dec. 19, 2005

Related U.S. Application Data
(63) Continuation-in-part of application No. 10/614,758, filed on Jul. 8, 2003.
Application No. 10/614,758 is a continuation-in-part of application No. 10/140,376, filed on May 8, 2002, now Pat. No. 6,669,481.
(60) Provisional application No. 60/636,615, filed on Dec. 17, 2004.
(60) Provisional application No. 60/289,116, filed on May 8, 2001.
(60) Provisional application No. 60/331,107, filed on Nov. 8, 2001.

(51) Int. Cl. G09B 19/00; A61B 5/00; A61B 19/00

(52) U.S. Cl. 434/236; 600/300; 128/897; 128/920; 434/118

(57) ABSTRACT

A system, method, and article of manufacture for providing automated neuropsychological testing. The invention includes a plurality of computer program modules wherein each program module includes computer readable instructions for administering a neuropsychological assessment test and a computer program control module that allows a user to control at least one parameter relating to the neuropsychological assessment test. The present invention also allows the user to build a custom-made test battery. The system of the invention includes a microprocessor capable of executing the computer program modules and the computer program control modules, an input device in communication with the microprocessor and an output device operable to provide information relating to each neuropsychological assessment test.

### I. CROSS-REFERENCE TO RELATED APPLICATIONS

This application claims the benefit of provisional application 60/636,615, filed on Dec. 17, 2004 and is a continuation-in-part of application Ser. No. 10/614,758 filed Jul. 8, 2003 which is a continuation-in-part of application Ser. No. 10/140,376 filed May 8, 2002, now U.S. Pat. No. 6,669,481, which claims the benefit of provisional application Ser. No. 60/289,116, filed May 8, 2001 and the benefit of provisional application Ser. No. 60/331,107, filed Nov. 8, 2001. Each of the foregoing are hereby incorporated by reference in their entireties.

### II. FIELD OF THE INVENTION

The present invention generally relates to neuropsychological testing, and more particularly to construction and administration of neuropsychological assessment tests.

### III. BACKGROUND OF THE INVENTION

As in almost every other field, the computer is heavily used in the neuropsychological testing field. Neuropsychologists, clinicians, and sometimes patients administer computerized neuropsychological tests for a variety of purposes. Computerized neuropsychological testing is ideal for a number of reasons. First, it allows for stimuli to be presented in a random form, thereby creating almost limitless combinations. Secondly, both individual and group testing may be accomplished with ease. Thirdly, the administrator can administer the tests, and, in some cases, interpret test results without the need for formal neuropsychological training. Finally, computerized neuropsychological testing results in a significant cost and time savings.

An early cognitive test system was the microcomputer-based testing system (MTS), an Apple® II-based system created to test cognitive skills. The system was developed in cooperation with the United States Environmental Protection Agency (EPA). It was used to test subjects for cognitive functions such as perception, reaction time, reasoning and decision making, and memory.

Perhaps, one of the most famous computerized neuropsychological testing packages is the Automated Neuropsychological Assessment Metrics (ANAM) system, a system of batteries of cognitive tests modified by neuropsychologists in the U.S. Armed Forces for precise measurement of cognitive processing efficiency. ANAM has been used to test subjects for sustained concentration and attention, mental flexibility, spatial processing, cognitive processing efficiency, mood, arousal/fatigue level, and short-term, working, and long-term memory.

Non-computerized neuropsychological tests have certain shortcomings such as restrictions on the number of combinations of stimuli presentations available during administration and limits on the speed of test administration. In addition, it is difficult for a person having limited neuropsychological training to reliably administer.

Known computerized neuropsychological tests, however, also have shortcomings. A shortcoming of some currently available computerized tests is that many of the computerized tests offer complex results, and the test administrator may exhaust a significant amount of time analyzing the results. The currently available neuropsychological computerized testing packages do not offer a simplified version of the results of each test to assist the test administrator's quick analysis of the test results.

Another shortcoming with some of the currently available computerized neuropsychological testing programs is that control of the content of the various tests is limited, as many of the control parameters are fixed. For example, many of the computerized neuropsychological tests employ control parameters such as a time value representing a length of time a particular stimulus is to be displayed to a user and a time value representing the length of time a user is given to respond to the stimulus. The time values may require adjustment to accommodate the various types of test subjects. For instance, an elderly person may have slow reflexes and require more time for the stimulus to be displayed and/or more time to respond to the stimulus. To alter the values associated with any aspect of a test, computer programmers must alter the code of the computer-implemented neuropsychological tests every time a change in the timing is desired.

Still another shortcoming with many currently available compute-implemented neuropsychological tests is the inability to choose a combination of neuropsychological tests to be executed (that is, test batteries). For example, in testing for dementia, a group of individual tests are believed to be superior by the neuropsychological community. The currently available computerized neuropsychological test packages, however, do not allow the user to create a test battery that includes a specific combination of tests used to assess the condition. For example, many computer-implemented neuropsychological tests display a predetermined font type and size as well as a predetermined stimuli size. Some test subjects have trouble seeing the test content (that is, the stimuli used in a particular test or instructional text) well enough to perform, as they should. This sometimes results in misleading test results.

In addition, some test subjects are legally blind and cannot view the text-based instructions to interact with the computerized test. Further, some test subjects are illiterate or are children who have not learned to read and therefore, cannot be presented with the test instructions in a visual format (for example, text-based instructions). Further still, for the children who have learned to read, their attention span is typically less than that of adults. As a result, children may become bored easily with simple text-like instructions. Thus, they may not pay close attention to the instructions, and their performance on the computerized tests may be compromised.

A further shortcoming of known compute-implemented neuropsychological tests is that test instructions are sometimes confusing. Thus, a test subject may have difficulty understanding the instructions, and an administrator of the test cannot be sure that a subject's poor performance on a test is actually a true assessment of his neuropsychological ability or merely an undesirable result of his failure to understand the instructions for the test.

In addition to the above content-related shortcomings of currently available computer-implemented testing packages, currently available computer-implemented tests suffer from set-up or test configuration problems. For example, computer system software packages are notorious for requiring more than one auxiliary file (for example, a data file). In many situations, these supplemental files are stored in a variety of locations (for example, different directories, different machines, different networks). These files are used by the main computer program, and if their content or location is altered, it can significantly degrade performance.

In light of the many shortcomings discussed above, what is needed is a computer-implemented neuropsychological testing system, method, and article of manufacture for allowing a user to control a variety of parameters of a computer-implemented neuropsychological assessment tests. Further, what is needed is a computer-implemented neuropsychological testing system, method, and article of manufacture that a person having little or no programming skill, for example, a clinician, researcher, or test administrator, can readily administer.

### IV. SUMMARY OF THE INVENTION

Given the following enabling description of the drawings, the system and method of the present invention should become evident to a person of ordinary skill in the art. The present invention encompasses a system, method, and article of manufacture for presenting one or more automated neuropsychological assessment test to a test subject.

The system of the present invention includes a plurality of computer program modules wherein each program module includes computer readable instructions for administering a neuropsychological assessment test, a computer program control module which allows a user to control at least one parameter relating to the neuropsychological assessment test, a microprocessor for executing the computer program modules and the computer program control module, an input device in communication with the microprocessor and capable of allowing the user to interact with the program modules, and an output device in communication with the microprocessor, the output device operable to provide information relating to each neuropsychological assessment test.

The present invention also encompasses a method for administering at least one neuropsychological assessment test. The method includes the steps of displaying a menu including names of neuropsychological assessment tests, receiving a selection relating to a neuropsychological test to be executed, allowing control of at least one parameter relating to the neuropsychological test to be executed, and executing the specific neuropsychological test in accordance with at least one parameter. In addition to the system and method briefly described above, the present invention presents an article of manufacture including a computer usable medium having computer readable program code means for causing a computer to execute at least one of a plurality of computer program modules.

The computer program modules include computer readable instructions for administering a neuropsychological assessment test. The article of manufacture also includes a computer readable program code means for causing the computer to allow a user to control at least one parameter relating to the neuropsychological assessment test. In at least one embodiment, the present invention allows the user to create a custom-made test battery.

### V. BRIEF DESCRIPTION OF THE DRAWINGS

Like reference numerals in the figures represent and refer to the same element or function.

FIG. 1 is a block diagram depicting an exemplary computer system capable of executing the computerized neuropsychological testing program modules of the present invention.

FIG. 2 is an illustration of an exemplary general information display screen according to an embodiment of the present invention.

FIG. 3 is an illustration of an exemplary display screen depicting information related to a specific test.

FIG. 4 is an illustration of an exemplary display screen depicting configuration information for a testing session according to an embodiment of the present invention.

FIG. 5 is a flow diagram illustrating the steps involved in a specific type of test of the present invention.

FIG. 6 is a flow diagram illustrating the steps involved in a specific type of test of the present invention.

FIG. 7 is a flow diagram illustrating the steps involved in a specific type of test of the present invention.

FIG. 8 is a diagram illustrating the composition of an exemplary series of test batteries according to an embodiment of the present invention.

FIG. 9 is a flow diagram illustrating the steps involved in an exemplary method of administering a computerized neuropsychological test battery of the present invention.

### V. DETAILED DESCRIPTION OF THE INVENTION

### Table of Contents

A. Overview
B. System
C. Test Administration
D. Library & Individual Tests
E. Battery of Tests
F. Battery Builder
   A. Overview

The present invention relates to a system, method, and article of manufacture for presenting automated neuropsychological assessment tests to a test subject. The invention offers a variety of features. For example, a library of automated individual neuropsychological testing program modules can be run in the present invention. For example, a test administrator may be presented with a library including a plurality of automated individual neuropsychological assessment tests. Each individual test may be configured by controlling a variety of parameters or variables (for example, a time for allowing stimuli to remain displayed). Some of the individual tests are enhanced versions of tests from the standard ANAM test battery. For example, the Sternberg Memory Search test has been enhanced to include a control feature that allows the test administrator to include a control to set a minimum time for viewing a stimulus set. Others of the tests are newly created neuropsychological tests. Further, the present invention has multi-media functionality useful for broadening the use of the computerized neuropsychological testing program modules. For example, audio and/or animated instructions can be used.

Further, the present invention includes a feature in which neuropsychological testing personnel (NTP) can create their own unique combination of individual neuropsychological tests. As used herein, the phrase “neuropsychological testing personnel” refers to clinicians, researchers, programmers, test administrators, test subjects and others involved in the testing process. For example, if the test subject is being assessed for a specific condition, the present invention presents the NTP with an option in which they can choose a specific combination of individual assessment tests from the plurality of neuropsychological assessment tests in the library (that is, from the plurality of computer program modules). In this way, the NTP can create test batteries specifically designed for given conditions.

In addition to offering the NTP the ability to create unique batteries of neuropsychological assessment tests, the present invention also allows the NTP the option of altering specific parameters or switches for each test within the battery. Thus, the NTP is not bound by preconceived program constraints, as they have full control of the neuropsychological tests via options such as controlling the individual parameters of each test, the order of execution of the tests of the battery, the number of times each test within the battery is run, whether instructions are displayed for each test, and what language is displayed for each test.

In some embodiments, the present invention further includes a menu program module that, transmits a common group of variables to one or more tests in the test battery, and allows the NTP to select a pre-constructed test battery for administration.

As described herein, the invention can take the form of an entirely hardware embodiment, an entirely software embodiment or an embodiment containing both hardware and software elements. In a preferred embodiment, the invention is implemented in software, which includes but is not limited to firmware, resident software, microcode, etc.

Furthermore, the invention can take the form of a computer program product accessible from a computer-usable or computer-readable medium providing program code for use by or in connection with a computer or any instruction execution system. For the purposes of this description, a computer-usable or computer readable medium can be any apparatus that can contain, store, communicate, propagate, or transport the program for use by or in connection with the instruction execution system, apparatus, or device.

The medium can be an electronic, magnetic, optical, electromagnetic, infrared, or semiconductor system (or apparatus or device) or a propagation medium. Examples of a computer-readable medium include a semiconductor or solid state memory, magnetic tape, a removable computer diskette, a random access memory (RAM), a read-only memory (ROM), a rigid magnetic disk and an optical disk. Current examples of optical disks include compact disk—read only memory (CD-ROM), compact disk—read/write (CD-R/W) and DVD.

A data processing system suitable for storing and/or executing program code will include at least one processor coupled directly or indirectly to memory elements through a system bus. The memory elements can include local memory employed during actual execution of the program code, bulk storage, and cache memories which provide temporary storage of at least some program code in order to reduce the number of times code must be retrieved from bulk storage during execution.

Input/output or I/O devices (including but not limited to keyboards, displays, pointing devices, etc.) can be coupled to the system either directly or through intervening I/O controllers.

Network adapters may also be coupled to the system to enable the data processing system to become coupled to other data processing systems or remote printers or storage devices through intervening private or public networks. Modems, cable modem and Ethernet cards are just a few of the currently available types of network adapters.

B. System

As briefly described above, according to at least one embodiment of the present invention, an automated neuropsychological testing system is provided that includes a plurality or library of computer program modules wherein each program module includes computer readable instructions for administering a neuropsychological assessment test. Exemplary neuropyschological tests include: The Modified Stanford Sleepiness Scale, The Mood Scale 2-R, The Simple Reaction Time Test, The Memory Search Test, The Running Memory and Continuous Performance Test, The Mathematical Processing Test, The Digit Set Comparison Test, The Logical Reasoning—Symbolic Test, The Code Substitution and Memory Test, The Spatial Processing Test, and The Matching to Sample Test. As is understood by the skilled artisan, other neuropsychological tests may be included in the library in accordance with the invention.

While each test in the library is unique, there are also several common attributes to each test. As mentioned above, the system also includes a computer program control module which allows the NTP, and, in certain instances the test subject, to control at least one parameter relating to a neuropsychological assessment test. Such parameters include a STIM_DURATION switch, a TRIAL_TIMELIMIT switch, a RANDOM_SEED switch, a TARGET_SPEED switch, a RESPONSE_DEVICE switch, a STIMULUS_SET switch, an EYE switch, an ISI_LEVEL switch, a SUB_BLOCKS switch, and a SET_SIZE switch, all of which will be described further below.

The system further includes a microprocessor or plurality of microprocessors for executing computer program modules and computer program control modules of the present invention. In keeping with the invention, the microprocessor may be housed in a desktop computer, laptop computer, palmtop computer, or personal digital assistant (PDA) or the like. Exemplary stand-alone computers may include, but are not limited to, Apple®, Sun Microsystems®, IBM®, or IBM®-compatible personal computers. Accordingly, the present invention may be carried out via a single computer system, such as a desktop computer or laptop computer. Preferably, the present invention is implemented using an IBM®-compatible computer system having minimum specifications of a Pentium® 90 MHz microprocessor, at least 32 MB RAM, and at least 4 MB free disk space.

The present invention is preferably implemented using the Windows 95/98/2000/NT4.0 or XP operating systems. In addition to the microprocessor, the present invention preferably utilizes an input device in communication with the microprocessor and capable of allowing a user to interact with the computer program modules. For example, in at least one embodiment, the present invention employs a keyboard as an input device. A computer mouse, lightpen, or touchscreen, for example, may also be employed with the present invention. An output device in communication with the microprocessor may also be utilized to provide information relating to a neuropsychological assessment test. Suitable output devices may include, for example, a computer display or monitor and/ or a computer speaker. The input device and the output device may comprise the same physical structure, e.g., when the input device is a touch screen.

In some embodiments, the system further preferably includes a statistical program module for providing test statistics such as mean, median, reaction time, accuracy, and throughput. Where the output device of the system is a display, each program module may include computer readable instructions for adjusting font of the textual information displayed thereon. In addition, the system may include a multimedia program for providing multimedia functions such as audio, animation, etc.

Referring to FIG. 1, an exemplary computer system 100 for implementing the automated neuropsychological program modules of the present invention is shown. The computer system 100 includes one or more processors, such as processor 104. The processor 104 is connected to a communications bus 106. It should be noted, however, that the processor 104 can also be connected to a crossover bar or another network. It should also be noted that various software embodiments are described in terms of the exemplary computer system depicted in FIG. 1. After reading the description herein, it will become apparent to a person skilled in the relevant art how to implement the invention using other computer systems and/or computer architectures.

Computer system 100 may include a communications interface 124 that forwards graphics, text, and other data from the communications bus 106 for display on a display device (not shown).

Computer system 100 also includes a main memory 108, preferably random access memory (RAM), and may also include a secondary memory 110. The secondary memory 110 may include, for example, a hard disk drive 112 and/or a removable storage drive 114, representing a floppy disk drive, a magnetic tape drive, or an optical disk drive, etcetera. The removable storage drive 114 reads from and/or writes to a removable storage unit 118 in a manner well known to those skilled in the art. Removable storage unit 118, represents a floppy drive, magnetic tape, memory stick, or optical disk. As will be appreciated by those skilled in the art, the removable storage unit 118 includes a computer usable storage medium having stored therein computer software and/or data.

In alternative embodiments, secondary memory 110 may include other similar means for allowing the neuropsychological test computer program modules of the present invention to be loaded into computer system 100. Such means may include, for example, a removable storage unit 118 and an interface 121. Examples of such may include a program cartridge and cartridge interface (such as that found in video game devices), a removable memory chip (such as an EPROM or PROM) and associated socket, and other removable storage units 122 and interface 121 which allows software and data to be transferred from the removable storage unit 122 to the computer system 100.

Computer system 100 may also include a communications interface 124. The communications interface 124 allows software and data to be transferred between the computer system 100 and external devices. Examples of the communications interface 124 include a modem, a network interface, an Ethernet card, a communications port, and a PCMCIA slot and card. Software and data transferred via the communications interface 124 are in the form of signals 128, which may be electronic, electromagnetic, optical, or other signals capable of being received by the communications interface 124. The signals 128 are provided to the communications interface 124 via a communications path (for example, a channel) 126. The path 126 carries signals 128 and may be implemented using wire, cable, fiber optics, a phone line, a cellular phone link, and RF link and other communications channels.

In accordance with an embodiment of the invention, the neuropsychological testing program modules of the present invention may be stored in main memory 108 and/or secondary memory 110. The neuropsychological testing program modules may also be received via communications interface 124. Such program modules, when executed, enable the computer system 100 to perform the features of the present invention as discussed herein. More specifically, the neuropsychological testing program modules of the present invention, when executed, enable the processor 104 to perform at least some of the features of the present invention. Accordingly, such computer program modules represent controllers of computer system 100.

In an embodiment where the invention is implemented using software, the software may be stored in a computer program product or an article of manufacture and loaded into the computer system 100 using removable storage drive 114, hard drive 112, or communications interface 124. Alternatively, the invention may be realized as computer program instructions embodied in a propagated signal. The control logic (for example, software) of the present invention, when executed by the processor 104, causes the processor 104 to perform the functions of the invention as described herein.

In other embodiments, the present invention is implemented primarily in hardware using, for example, hardware components such as application specific integrated circuits (ASICs). Implementation of a hardware state machine so as to perform the functions described herein will be apparent to persons skilled in the art. The functionality of the neuropsychological testing program module(s) of the present invention will now be described herein.

C. Test Administration

In at least one embodiment of the present invention, a method in a computer system for administering at least one neuropsychological test is provided. The method includes the steps of displaying a menu including names of neuropsychological assessment tests, receiving a selection relating to a neuropsychological assessment test to be executed, allowing control of at least one parameter relating to the neuropsychological test to be executed, and executing the specific neuropsychological test in accordance with the at least one parameter.

In an exemplary embodiment of the present invention, a computer program product containing a computer usable medium is provided. The medium has computer readable program code means for causing the computer to execute at least one of a plurality of computer program modules, wherein each program module includes computer readable instructions for administering a neuropsychological assessment test. One skilled in the art will recognize that the computer readable instructions can be in the form of any viable computer programming language. For example, a high-level programming language such as C, C++, Fortran, or Beginners All-Purpose Symbolic Instruction Code (BASIC) are believed to be suitable to program the program modules of the present invention. It should be noted that the term “program module” or “program control module” is used herein to refer to a set of computer instructions for accomplishing a task. Thus, as used herein, a program module may be embodied in a single electronic file or medium or in multiple files or media.

An article of manufacture in accordance with the exemplary embodiment includes a first computer readable program code means for controlling at least one parameter relating to a neuropsychological assessment test responsive to user input. In addition to the computer readable code means for causing the computer to execute at least one of a plurality of computer program modules, the article of manufacture of the present invention preferably includes a computer readable program code means for causing the computer to execute a statistical program module for providing statistics (for example, mean, median, reaction time, accuracy, and throughput). The article of manufacture preferably further includes computer readable program code means for causing the computer to execute a multimedia program module for providing multimedia function (for example, audio user instruction sets wherein each user instruction set corresponds to a neuropsychological assessment test). In at least one embodiment, the multimedia program module utilizes user interface enhancement software. Each program module further facilitates audio testing administration.

In addition to the program modules of the present invention, in at least one embodiment of the present invention, a menu program module is provided to facilitate selection of a neuropsychological assessment test. In addition to offering a choice of neuropsychological assessment tests, the menu program module preferably generates a display screen including general information relating to the module including but not limited to the title, sponsors, version revision, authors, and creators, as illustrated in FIG. 2, for example.

The menu program module may also create a general information screen that presents information relating to an individual test including but not limited to the title of the test and the purpose of the test. For example, upon selecting a neuropsychological assessment test, the user may be presented with the display screen depicted in FIG. 3. The exemplary display screen depicted in FIG. 3 displays a test name, and the purpose of the test. As illustrated in FIG. 4, in addition to presenting the above, the menu program module of the present invention also preferably prompts the user for a series of variables including a subject ID code, the type of test, whether instructions are to be presented for the particular test, a specification location for the instruction file, a run number, a dominant hand indication, and the type of run, for example. It should be understood that these variables are meant to serve as examples, and the present invention may offer other variables in its various embodiments. The above-mentioned variables will now be described:

Subject Identification:

The Subject Identification “ID” variable identifies the test subject. For example, the ID may be a four-digit code including letters and/or digits. Common Subject ID codes include the last four digits of the patient's social security number.

Type of Test:

The Type of Test variable indicates whether a test to be executed is a practice test administration or a formal test administration.

Instructions:

The Instructions variable determines whether instructions are to be displayed for the test to be executed. (For example, if instructions are desired for a test, a user may enter ‘Y’ for “yes” or ‘N’ for “no.” Similarly, the user may select a “yes” or “no” radio button, for example, to indicate whether instructions are desired.

Instruction File Extension:

The Instruction File Extension variable determines the language of choice for display of user test instructions. For example, an extension of “IN0” preferably indicates that the instructions are to be displayed in the English language as opposed to the Spanish language or some other language.

Run Number:

The Run Number variable indicates the test session and is automatically incremented to count the test sessions.

Dominant Hand:

The Dominant Hand variable indicates which hand of a test subject is the dominant hand. For example, if the test subject is right-handed, an ‘R’ may indicate that he is right-handed. Similarly, if the test subject is left-handed, an ‘L’ may indicate that he is left handed.

Type of Run:

The Type of Run variable indicates the number of tests to be run. For example, an ‘S’ indicates that a single test is to be run.

After the response to each inquiry is entered, the menu program module displays the contents of each variable and provides the NTP with an opportunity to alter the contents of each variable. For example, FIG. 4 depicts a display screen illustrating the variables and exemplary contents of each.

After displaying the contents of the variables, the menu program module preferably “asks” the NTP if the variables are as desired and provides the NTP with an opportunity to alter the contents of the variables. In addition to prompting for the above, the menu program also prompts the NTP for subject information and demographics. It should be noted, however, that in at least one embodiment of the present invention, a program module other than the menu program module may be responsible for collecting subject information and demographics (for example, a subject information collection module). The subject information and demographics information includes but is not limited to age, sex, race and occupation of the test subject, years of education, how many times the test subject has been tested, and the exact tests that the test subject has undergone for example. The subject information and demographics module may also include the subject ID and the name of the test or battery and any other pertinent information.

The present invention may also employ a demographics module for gathering personal information from a test subject. For example, using the present invention, the NTP is presented with a menu through which he can elect to enter demographic information concerning the test subject such as the subject's age, occupation.

The program modules of the present invention allow the NTP to accommodate a variety of types of test subjects. The present invention employs a variety of user-friendly features that assist in ensuring reliability and accuracy of test results. Many of the program modules of the present invention employ user instruction sets for providing detailed instructions of exactly how the test subject should interact with a particular testing program module. For example, a particular neuropsychological test may generate a snowflake that flashes on the screen at a given period and frequency. The test subject preferably employs an input device to respond to test prompts or queries. For example, where the input device is a mouse, the test subject preferably depresses one of the buttons to input its test responses. Thus, it is desirable to present the test subject with instructions regarding which computer mouse button should be pressed. The present invention also allows the NTP to adjust the font type and size of the text-based instructions presented to the user. After a user selects the particular program module from a presented menu, the user is then presented with an option in which he can choose to change the default font for user instructions. For example, if the instructions informing the test subject to depress the left mouse button every time the snowflake flashes are presented in the “Times New Roman” font type with a size of 12 point, the test administrator can elect to change the font type to “Courier” with a size of 14 point by electing a “change font” option included within the particular program module. Upon selection of the option, the user of the program module, is presented with a plurality of font types and sizes from which he may choose to have instructions displayed. Alternatively, the user may be allowed to enter the font information. Such a feature is useful for allowing visually impaired test subjects to see the instructions displayed on a computer display screen more clearly.

Just as the user of the computer program modules of the present invention can change the particular font type and size for user instructions, the user may also change the contrast, brightness, background colors, and font type and size for text that may be displayed throughout execution of the program without adjusting the output device (display). For example, the present invention also presents the user with an option of altering the contrast control for the flashing snowflake in the example above. Similarly, the background of a particular test may be modified to display graphical images such as a bitmapped image or any other viable graphical image.

Another user-friendly feature of the present invention also relates to user instructions. While in some embodiments standard text-based instructions are presented to the test subject, in other embodiments, the program modules of the present invention present audio-based instructions to the test subject. Audio instructions are particularly useful for assisting visually impaired test subjects. Thus, continuing with the snowflake example offered above, instead of seeing the instructional text on the screen, the test subject would be presented with sound reciting how to respond to the flashing snowflake.

In addition to providing audio instructions for the tests, the present invention provides multi-media instructions. For example, in accordance with an aspect of the invention, the program module presents the test subject with a combination of visual animated graphical instructions and audio instructions. User interface enhancement software such as Microsoft Agent® can be utilized to assist in providing multi-media capability to the test subject. As those skilled-in the art will appreciate, software such as the Microsoft Agent package allows animated characters to be displayed on a screen and interact with the user to provide a more appealing and exciting presentation of user instructions. Such functionality is ideal for younger test subjects such as children, as they tend to quickly become bored with traditional text-based instructions. Further, it should be noted that the present invention also provides audio-based testing for some of the program modules. For example, some of the program modules do not require visual presentations. The present invention can offer these program modules to the test subject in an audio-based format.

Ensuring that the test subject understands the user instructions presented is important, as failure to understand the user instructions can cause the test subject to perform poorly on a particular neuropsychological test. Consequently, the test results will not represent the true ability of the test subject, and any assessment may be compromised. Therefore, the present invention provides an error-checking feature for ensuring that the test subject understands the user instructions provided. For example, a particular test may present a test subject with several columns with each column having a number and a matching symbol. After presenting the test subject with the combinations of numbers and symbols pairing, the test may require the test subject to indicate whether a given symbol and number combination matches any of those displayed in the columns previously presented on a computer display screen.

If the test subject responds incorrectly a specified n number of times, according to embodiments of the present invention, a message will be displayed to him informing him that he may have misunderstood the user instructions initially provided. The test subject is then presented with a different wording of the initial instructions and allowed to begin the test anew. In embodiments of the present invention, the value of “n” may be modified to accommodate the expectations for each test and/or each type of test subject. For example, the value of the variable “n” could be specified to be higher for children than for adults, as children may be expected to have more errors on the test than adults. Thus, their repeated errors may not, in fact, be an indication that they do not understand the user instructions.

In addition to providing a user-friendly content environment, the present invention provides user-friendly results of the various neuropsychological tests. For example, the present invention provides standardized test result data for the neuropsychological tests in the library. Thus, the computer modules of the present invention generate data including mean/median, reaction time, accuracy, and throughput. After selection of a particular test, the NTP may be offered an option through which he can elect to display the results of the test including the data described above.

In addition to the user-friendly features described above, the present invention also provides a data-securing feature that prevents undesired tampering with data. This feature includes the capability to “lock-out” the test subject, thereby preventing him from changing test configuration information such as the storage area of data files (for example, user instruction files), period and frequency of stimuli display and whether instructions will be displayed. For example, a set of user instructions for a particular program module may be stored in a separate location from the actual program module. To prevent a user from altering these instructions, using the present invention, a system administrator can designate this area “off-limits” to certain individuals. Thus, the integrity of this data is not compromised.

In certain situations, however, it may be necessary to allow changes to this information. For example, the user instruction sets for a particular program module may require updating. Thus, at least one individual (for example, a system administrator) requires access to the area in which the information is stored. To accommodate this designated individual, the present invention includes a special code that can be utilized to temporarily gain access to the storage area containing the secured information.

The present invention accomplishes this feature by providing the user with a menu choice through which he is prompted for a special pass code or key. Upon entering the correct pass code or key, the user is granted access to the area in which the secured information (for example, the text of the user instructions) is stored. If, however, the user enters an incorrect pass code or key, he is presented with a message indicating that the key he entered does not match the valid key or pass code.

Yet another feature of the present invention is the ability to perform automated configuration management of the software (that is, the computer instructional code of the neuropsychological testing program modules) of the present invention. For example, in embodiments of the present invention, it is less desirable to execute a program module if it is not the latest version. Upon choosing an option to execute a particular program module of the present invention that is not the latest version, an informational message is preferably displayed informing the viewer that there is a more recent version of the program module available. Such a feature helps ensure that the software remains up-to-date. Thus, the test administrators achieve the maximum possible benefit from assessment of test subjects using the present invention.

In some embodiments, the above-discussed “dating” feature of the present invention is accomplished by encoding a date code directly into the menu program module (that is, the set of computer instructions that causes a menu of choices to be displayed to the user) or the computer instructional code of each program module. Thus, upon attempted execution of a particular program module, the encoded date code is compared to a date stamp of the system on which the program is loaded. If the system date stamp is earlier in time than that of the encoded date stamp, then the particular program module is executed. If, however, the system date stamp matches the encoded date code or is later in time than the encoded date code, execution of the particular program module is not allowed, as the program is outdated. In such a situation, a message is presented to the user informing him that program module execution is not allowed because the module is outdated. This feature provides facilitates purging of outdated desktop software

In other embodiments, the above-discussed dating feature of the present invention is accomplished by storing a date value in a variable or parameter of the menu program. In these embodiments of the present invention, rather than encoding the date code directly into the menu program or directly into each program module, the date code is stored in a variable or parameter in the menu program module Access to this variable or parameter can be altered by a designated individual and protected by the “lock-out” feature described above. In these embodiments, the date code can be changed whenever a new version of a particular program is received. It should be noted that each program module can have a date code, or all programs may share one date code for operation.

As discussed above, many of the program modules provide complex results for analysis by the NTP. In many instances, the NTP may become overwhelmed by the complexity of the test results. In situations involving execution of a battery of tests (described below), this can be extremely overwhelming to the NTP. The present invention assists in minimizing this effect through its View program module. The View program module of the present invention provides the NTP with a quick synopsis of the results of a particular test or the results of a battery of tests. In embodiments of the present invention, test results can be displayed in graphical form. In addition, the NTP may select the data items for which he would like to receive a report, as well as the tests for which he would like to receive results. Further, the NTP can elect to receive recommendations on how he should proceed with a particular test session such as what testing should follow.

For example, after executing a test, the NTP can select the View option to display a quick synopsis of the results of the particular test. If, for example, the test subject performed very poorly on a particular test, the present invention presents the NTP with a plan and/or suggestion for what tests should follow to further access the weaknesses of the test subject.

D. Library & Individual Tests

In keeping with the invention, a library of neurocognitive test modules is provided for measuring performance degradation from injury, illness, fatigue, medications, chemical exposure, radiation or for measuring any other perceived cognitive loss or gain. The library includes several known neuropsychological tests that have been modified for computer implementation as well as some newly developed neuropsychological tests. The tests include but are not limited to the following: 1) The Modified Stanford Sleepiness Scale, 2) The Mood Scale 2-R, 3) Simple Reaction Time Test, 4) Memory Search Test, 5) Running Memory and Continuous Performance Test, 6) Mathematical Processing Test, 7) Digit Set Comparison Test, 8) Logical Reasoning—Symbolic Test, 9) Code Substitution and Memory Tests, 10) Spatial Processing Test, and 11) Matching to Sample Test. While each test in the library is unique, there are also several common attributes to each test.

FIG. 5 is a flow diagram illustrating the general steps of an exemplary method of execution for a specific type of test according to an embodiment of the present invention. More specifically, FIG. 5 illustrates the steps involved in a test that presents an on-screen query to the test subject. One of the more popular tests of this type is the Modified Stanford Sleepiness Scale Test, which will be specifically described in further detail below.

Referring now to FIG. 5, flow begins with step 505 and proceeds to step 510. In step 510, a list of switches (parameters) described below is generated. In some embodiments, the NTP may have sufficient privileges to select and set one or more of the switches to configure the tests as desired. In other embodiments, only the system administrator has sufficient privileges to alter switches.

In step 515, the name of the test is preferably displayed on the display screen. Control then proceeds to decision step 520. In decision step 520, the program module prompts the user to determine whether she would like instructions for the test. If the user would like instructions for the test, in step 525, the program module communicates a set of user instructions for taking the test. Returning to decision step 520, if the user elects not to have user instructions displayed, an initial start screen is presented, for example, an introductory screen with graphics.

In step 535, control threads are set. In step 540, the display screen is cleared. In step 545, stimuli, e.g., statements for the test, are displayed. For example, the Modified Stanford Sleepiness Scale Test comprises seven descriptive statements used to describe how the test subject feels with respect to alertness or sleepiness. The statements may be displayed to the test subject one at a time or all statements may be displayed simultaneously.

In step 550, the test subject's response to the presentation of the statements presented in previous step 545 is received. For example, the test subject may select one of the statements by highlighting the statement or clicking a radio button corresponding to the particular statement of selection.

In step 555, the test subject's response to the statements is recorded. In step 560, a report is created that includes the test subject's responses to the statements. The report may be displayed or stored for later viewing. Finally, control ends with step 565. The following are examples of neuropsychological tests of the type described with respect to FIG. 5.

Modified Stanford Sleepiness Scale

This test is comprised of a series of statements that describe how one feels with respect to alertness or sleepiness. In a preferred embodiment, this test comprises seven descriptive statements. The program module may display the statements to the test subject one at a time or all simultaneously. In any event, the test subject is directed to select the statement that best describes its feelings at that moment in time. The test subject may make the selection on screen using a mouse, the keyboard or any other known input device. The seven descriptive statements of the preferred embodiment are:
Feeling very alert, wide-awake, and energetic.
Able to concentrate, but not quite at peak.
Relaxed, awake, responsive, but not fully alert.
A little foggy and mild difficulty concentrating.
Foggy, slowed down, beginning to lose interest in remaining awake.
Sleepy, woozy, prefer to be lying down; fighting sleep.
Sleep onset soon, losing struggle to remain awake.
   Mood Scale 2-R

This test is designed to assess either mood state or trait in test subjects in six subcategories that include Activity (high energy-level), Happiness (positive disposition), Depression (dysphoria), Anger (negative disposition), Fatigue (low-energy level), and Fear (anxiety level). The test includes a plurality of subscales, each including a plurality of adjectives.

In operation, the test subject is provided with an on-screen query and a variable response and indicia for the test subject to indicate either agreement or disagreement with the response. In preferred embodiments, the query remains constant for each subscale and the response varies among the plurality of adjectives linked to the subscale. Participants are asked to reply to the variable response by selecting the appropriate indicia, for example, by pressing 1, 2, or 3 on the computer keyboard, (that is, “press 1 for yes, 2 for somewhat, and 3 for no”) in response to the query, “How does the word shown below describe how you feel right now?” Once the participant has selected the appropriate indicia, the variable response is changed and the participant replies to the updated variable response. Scores for each of the six scales are produced and stored in a participant database.

The adjectives or responses linked to the Activity subcategory include energetic, lively, alert, spirited, active and steady. The adjectives linked to the Happiness subcategory include good, content, cheerful, satisfied, pleased and happy. The adjectives linked to the Depression subcategory include miserable, discouraged depressed, sad, downcast and gloomy. The adjectives linked to the Anger subcategory are grouchy, enraged, annoyed, angry, furious and irritated. The adjectives linked to the Fatigue subcategory include inactive, weary, drowsy, tired, sluggish, and lazy. The adjectives linked to the Fear (anxiety) subcategory include uneasy, alarmed, insecure, afraid, nervous and anxious. There are also other sets of adjectives which may be associated to these categories

FIG. 6 is a flow diagram illustrating the general steps of an exemplary method of execution for a specific type of test according an embodiment of the present invention. More specifically, FIG. 6 illustrates the steps involved in a type of test that presents a stimulus to the test subject. One of the more popular tests of this type is The Simple Reaction Time Test, which will be specifically described in further detail below.

Referring now to FIG. 6, flow begins with step 605 and proceeds to step 610.

In step 610, a list of switches (parameters) described below is generated. In some embodiments, the NTP may select and set one or more of the switches to configure the tests as desired. In other embodiments only the system administrator or his designee has sufficient privileges to set and select switches.

In step 615, the name of the test is preferably displayed on the display screen. Control then proceeds to decision step 620. In decision step 620, the program module prompts the user to determine whether she would like instructions for the test. If the user would like instructions for the test, in step 625, the program module communicates a set of user instructions for taking the test. Returning to decision step 620, if the user elects not to have user instructions displayed, an initial start screen is presented, for example, an introductory screen with graphics.

In step 635, control threads are set. In step 640, the display screen is cleared. Control then passes to decision step 645.

In decision step 645, it is determined whether a count variable (for example, “Kount”) has reached the specified number of trials for ending the program module, or whether the specified time for the session is up. As used herein, a trial is defined as a series of stimuli presentations. That is, a plurality of stimuli may be presented to the test subject in a trial. Preferably, a session is comprised of one or more trials. Now referring back to FIG. 6, in accordance with a specific example, the total number of trials (for example, “ntrials,” in FIG. 6) for the program module may initially have been set to five, and the count variable (for example, “kount,” in FIG. 6) may now specify a value of three.

Continuing with the example offered above, if the current “kount” value (which indicates the current session number) is three and the “ntrials” (total number of trials initially set) variable is five, then there are two more trials left in the test to execute. Thus, control within the program module does not end and proceeds to step 650.

In step 650, the “kount” variable is incremented by one to reflect execution of yet another trial of the test. In other words, the current “kount” value is updated to reflect execution of the present trial.

In step 655, a group of variables is set for the current trial. For example, step 655 illustrates the setting of a default response variable, a gap variable, and an interstimulus variable. The interstimulus variable may indicate the time allowed between stimulus presentations. In step 660, a stimulus is presented on the display screen. Control then proceeds with decision step 665. In decision step 665, it is determined whether an input device has been activated. For example, a user may depress a key on the keyboard or computer mouse to respond to the stimulus as instructed by the Simple Reaction Time test. If it is determined that the test subject has activated an input device in decision step 665, control resumes with step 670, and the test subject's response to the stimulus is recorded. In step 675, the length of time it took the test subject to respond to the test is also recorded.

Returning to decision step 665, if it is determined that the user did not activate the input device, control resumes with step 640, where the display screen is cleared. Returning to decision step 645, if the current trial number (that is, the “kount” variable) has reached the number stored in the “ntrials” number (or the time allotted for testing has expired), no further trials of the program module will be executed. Control then resumes with step 680.

In step 680, the statistics for the test subject's performance are calculated. In step 685, at least one report of the statistics is created. Control then proceeds with decision step 690, in which it is determined whether feedback (that is, performance results from the test) from the user's performance is to be displayed on the display screen. If it is determined that feedback from the user's performance is to be displayed on the screen, the feedback is displayed in step 695. If it is determined that feedback is not to be displayed in decision step 690, control of the testing program module ends in step 697. In some embodiments, it may be desirable to prevent the test subject from viewing the test results and to only allow the NTP to view the test results. It should be noted that a test subject may desire to save the report created in step 685 to a disk drive, for example, for later viewing. The following are examples of neuropsychological tests of the type described with respect to FIG. 6.

Simple Reaction Time

This is a test designed to provide a measure of pure reaction time, an important aspect of neurocognitive assessment. In operation, a simple stimulus is presented on screen, and the participant is instructed to input a response each time the stimulus is presented. When the stimulus is presented, it remains on screen for a predetermined period of time hereinafter referred to as the RT period. The RT period may be adjusted as desired by the test administrator. For example, when testing a jet pilot it may be desirable to have a very short RT period that requires a quick response. However, when testing a subject who's reflexes are not as keen, for example, Alzheimer's candidates, it may be desirable to have a longer RT period, thus allowing slower response times. Similarly, the presentation rate of the stimulus may be varied as desired by the administrator.

2-Choice Reaction Time

This test is similar to the Simple Reaction Time Test except that two different stimulus characters are presented, for example, “+” and “*”. The pattern of presentation of the stimulus, the RT period and the rate of presentation may be adjusted by the NTP as desired.

Spatial Processing

In this test, a pair of four bar histograms is displayed as pairs, and the subject is requested to determine whether they are identical. One histogram is preferably rotated either approximately 90° or approximately 270° with respect to the other histogram. The subject may respond to indicate that the two histograms are either the same or different using the input device.

Running Memory (Continuous Performance Test)

This test is intended to index the test subject's concentration level and attention span. The test requires the subject to continuously compare characters, for example, numbers or symbols. In operation, characters are presented on screen one at a time, preferably in the center of the screen. In accordance with a preferred aspect of the invention, the characters are presented in a pseudo random order. Prior to being presented with the characters, test subjects are instructed to continuously monitor the characters and input a response using an input device if the character on screen matches the character that immediately preceded it. The test subject is instructed to input a different response, that is, press another key or button, if the character on screen does not match the character that immediately preceded it.

Each character may be displayed on the screen for a period of time that may be selected by the NTP prior to administration of the test. In addition, the test administrator may select/adjust the period of time between presentations of successive characters. For example, characters may be presented either after a predetermined time has elapsed or responsive to the test subject's response input, whichever occurs first. In preferred embodiments, characters should be displayed as described in connection with this test for about five minutes to effectively assess the subject's concentration level and attention span.

Symbolic & Procedural Reaction Time (Modified Stress rt Tasks)

In this test, a variety of characters are presented on screen, one at a time and preferably in the center of the screen. In a preferred embodiment, four distinct characters are presented. For example, the characters may include shapes or alpha-numeric characters. The test subject is instructed to input a first specified response each time first and second characters are detected and to input a second specified response each time third and fourth characters are detected. The test subject may input its response by depressing specified keys on a keyboard, by selecting a specified input using a mouse, or by using any other known input procedure.

In accordance with an aspect of the invention, in one embodiment, the character quality is normal and the characters are presented at regular intervals. In accordance with another embodiment, the character quality is degraded and the characters are presented at regular intervals. In accordance with still another embodiment, the character quality is normal but the characters are presented at irregular intervals. Advantageously, the test administrator may control the duration and period of the intervals without reconfiguring program code (that is, this is a user controllable parameter).

FIG. 7 is a flow diagram illustrating the general steps of an exemplary method of execution for a type of test that presents a stimulus and provides an option for a practice session. The Mathematical Processing Test is a common test of this type. Flow begins with step 705 and proceeds to step 310. In step 710, a list of switches (parameters) described below is generated. The NTP may select and set one or more of the switches to configure the tests as desired.

In decision step 715, the program module prompts the user to determine whether she would like user instructions for the test. If the user would like user instructions for the test, in step 720, the program module displays a set of user instructions instructing the user how the test works. Returning to decision step 715, if the user elected not to have user instructions displayed, in step 725, the name of the test is displayed, and an initial start screen is presented (for example, an introductory screen with graphics). In step 730, stimuli are presented. For example, the Mathematical Processing Test displays a mathematical problem to the test subject on a display screen. In particular situations, the stimuli or mathematical problems may be stored in an auxiliary file, for example.

In step 735, control threads are set. In step 740, the display screen is cleared. Control then passes to decision step 745.

In decision step 745, it is determined whether a count variable has reached the specified number of trials for ending the program module, or whether the specified time for the session is up. For example, the total number of trials (for example, “ntrials,” in FIG. 7) for the program module may initially have been set to five by the user, and the count variable (for example, “kount,” in FIG. 7) may now specify a value of three.

Continuing with the example offered above, if the current “kount” value is three (that is, the current trial is trial number three) and the “ntrials” (total number of trials initially set) variable is five, then there are two more trials left in the test to execute. Thus, control within the Mathematical Processing Test program module does not end and proceeds to step 750, providing that the time allotted for the session has not expired.

In step 750, the “kount” variable is incremented by one to reflect execution of yet another trial. In other words, the current “kount” value is updated to reflect execution of the present trial. In step 755, a group of variables is set for the current trial. For example, step 755 illustrates the setting of a default response variable, a gap variable, and an interstimulus variable. For example, the interstimulus variable may indicate the time allowed between stimulus presentations.

In step 760, a stimulus is selected by the user. In step 765, the stimulus is displayed on the display screen. Control then proceeds with decision step 770. In decision step 770, it is determined whether the test subject has generated input. For example, a user may depress a key on the keyboard or computer mouse to respond to the stimulus as instructed by the test. If it is determined that the test subject has generated input in decision step 770, control resumes with step 775, and the test subject's response to the stimulus is recorded. In step 780, the length of time it took the test subject to respond to the stimulus is recorded. Alternatively, in decision step 770, if it is determined that no input was generated, control proceeds to step 785.

Some tests are rather complex (for example, the Mathematical Processing Test) and thus may present the test subject with practice sessions in which they can learn how to interact with the tests. In such tests, the test subject is prompted to enter whether he desires a practice session after user instructions are displayed.

Thus, in decision step 785, it is determined whether the current session is actually a practice session. If it is determined that the current session is a practice session, in step 790, an encouraging graphical display (that is, some type of positive reinforcement) is presented on the display screen, for example, a “smiley” face. Returning to decision step 785, if it is determined that the current session is not a practice test (that is, if it is determined that the current session is an actual testing session), then control proceeds to step 740, where the screen is again cleared.

Returning to decision step 745, if it is determined that the value for the current trial is equal to the value of the total number of trials set, or if it is determined that the time allotted for the session has expired, then no more trials are to be executed.

In step 791, the statistics for the test subject's performance are calculated. In an optional step 792, at least one report of the statistics is created. Control then proceeds with decision step 793, in which it is determined whether feedback from the test subject's performance is to be displayed on the display screen. If it is determined that feedback from the test subject's performance is to be displayed on the screen, the feedback is displayed in step 795. If it is determined that feedback is not to be displayed in decision step 793, control of the test module ends in step 797. The following are neuropsychological tests of the type described with reference to FIG. 7.

Mathematical Processing

This test is used to assess the test subject's simple arithmetical and concentration abilities. In this test, arithmetic problems are presented on screen, preferably in the middle of the screen. The test requires the subject to deduce an answer and then decide if the answer is greater-than or less-than a specified number, for example, the number five. Each problem includes two mathematical operations (addition and/or subtraction) on sets of three single-digit numbers (for example, 5+3−4=?). The subject is instructed to read and calculate from left to right and indicate whether the answer is greater-than or less-than a given number, for example, five, by pressing one of two specified response buttons/keys. The operators and operandi are selected at random with the following restrictions: only the digits 1 through 9 are used; the correct answer may be any number from 1 to 9 except 5; greater-than and less-than stimuli are equally probable; cumulative intermediate totals have a positive value; working left to right, the same digit cannot appear twice in the same problem unless it is preceded by the same operator on each occasion (for example, +3 and +3 are acceptable, while +3 and −3 are not); and the sum of the absolute value of the digits in a problem must be greater than 5.

Digit Set Comparison

This test is used primarily to index immediate memory and attention. In this test, a string of digits ranging in length from 2 to 10 numbers is presented in the center of the screen. After a specified period, the first string of digits disappears and a second string is presented. The duration of the specified period is adjustable and may be controlled by the NTP without alteration of the program code (that is, this is a user controller parameter). The test subject is instructed to compare the two strings of digits and decide if they are the same digits and in the same order. The test subject is further instructed to respond by providing an input.

Logical Reasoning

This test is useful for indexing ability for abstract reasoning and verbal syntax. It is a linguistic task requiring knowledge of English grammar and syntax. It also requires the ability to determine whether various simple sentences correctly describe the relational order of two symbols. In this task, stimulus pairs may be presented one at a time and are preferably screen-centered rather than left justified to reduce differences in visual search times.

On each trial the symbol pair “# &” or “& #” is displayed along with a statement that correctly or incorrectly describes the order of the letters as depicted in the example below:
&#
# is first

The subject is instructed to decide as quickly as possible whether the statement is true or false and then to provide an input indicating the response.

Code Substitution and Memory Test

In this test, a string of preferably up to 9 symbols and 9 digits may be displayed across the upper portion of the screen and preferably arranged so that the digit string is immediately below the symbol string. There is one digit corresponding to each symbol. During the test, a test pair (a symbol and corresponding digit) may be presented at the bottom of the screen, below the digit/symbol string. The goal of the test is to determine whether the test pair matches any associated pair in the string. The test taker may respond using an input device to indicate that the test symbol and digit are a correct or incorrect pairing.

The initial presentation is a visual scanning and learning procedure. The ratio of correct to incorrect displays is preferably 3:1, and each pair is preferably presented a minimum of 8 times yielding at least 6 correct presentations and at least two incorrect presentations per digit symbol set.

An associative recognition memory trial is then presented immediately and at a selected time interval following the learning trial, the time interval being controllable by the NTP without modification of program code. During this portion of the test, the procedure is essentially the same as the learning procedure. However, the comparison coding strings are not displayed. Only the test stimuli are presented and the subject has to indicate whether the displayed pair is correct or incorrect based on the subject's recollection of the paired associates presented during the learning trial. The ratio of correct to incorrect presentations during the associative recognition trial is preferably approximately 1:1. So there is about a fifty percent chance of being presented with a correctly matched symbol and digit pair.

Matching to Sample

In this test, the subject is asked to respond to stimuli that correspond in some fashion to a sample stimulus. In a preferred embodiment, a 4×4 matrix (checkerboard) is initially presented in the center of the screen as a sample stimulus to the subject. For each trial presentation of a matrix, the number of cells that are shaded may be varied at random from only one cell to twelve cells. When the subject responds via the input device or after a predetermined period of time, for example, thirty seconds, the sample matrix is removed from the screen. Following a second predetermined time interval, for example, twenty seconds, a set of two comparison matrices are presented side by side on the screen. One of the comparison matrices matches the sample matrix and the other comparison matrix preferably differs in shading from the sample by one cell. The subject's task is to indicate using the input device, which matrix matches the sample matrix.

Memory Search (Sternberg RT & Symbolic)

This test is useful for indexing the subject's working memory. In accordance with the invention, a set of characters is displayed horizontally, preferably in the center of the monitor. The set of characters is referred to herein as the “memory set”. The test subject should view the memory set until it is memorized. The memory set is preferably comprised of 2, 4, or 6 letters and/or symbols. However, the number of characters that comprise the memory set may be varied by the administrator to accommodate the testing environment.

After the memory set has been learned, the subject generates an input, for example, presses a response key, to begin the test. During this part of the test, single “probe” letters or symbols are presented preferably in the center of the screen, and preferably one at a time. The subject indicates whether or not the probe matches any of the memory set items. Responses may be entered by pressing a specified key or mouse button. Each probe remains on the screen until the subject responds or until a pre-selected time limit has elapsed. The screen is cleared momentarily between successive probe presentations.

In keeping with a particularly preferred aspect of the invention, each successive administration of the test uses a unique memory set. Memory set letters are preferably selected randomly from the following list: A, B, C, E, F, G, H, I, J, K, L, M, Q, R, S, T, U, X, Y. Or the memory set could comprise digits or other symbols. The memory set characters are preferably centered horizontally in the middle of the screen with one character space between each letter. Positive probes, that is, probes that match one of the memory set characters, are equally likely to match any of the memory set letters. Further, positive and negative probes (probes that do not match any of the memory set characters) are presented in an equal-probability randomized or pseudo-randomized order. In constructing the memory set, it is desirable to exclude characters that are similar in appearance. For example, in the preferred embodiment, the letters U and V were expressly excluded from the memory set so as not to be mistaken, one for the other.

In addition to the various automated individual neuropsychological testing program modules, according to embodiments of the present invention, each neuropsychological program module may be customized by controlling a variety of parameters, switches, or variables relating to a variety of functions for each test. The switches include but are not limited to the following: (1) RESPONSE_KEYS, (2) STIM_DURATION, (3) HAND, (4) RESPONSE_DEVICE, (5) EYE, (6) PRACTICE_MODE, (7) STIMULUS_SET, (8) RANDOM_SEED, (9) TRIAL_TIMELIMIT, (10) SOUND_TOGGLE, (11) HEMISPHERE_ORDER, (12) SUB_BLOCKS, (13) ISI_LEVEL, (14) SET_SIZE, (15) NUMBER_OF_TRIALS, (16) TARGET_SPEED, (17) VIEW_DELAY.

RESPONSE_KEYS

The RESPONSE_KEYS switch specifies a keyboard string that denotes acceptable keys on the keyboard for user input. For example, if the RESPONSE_KEYS switch is A,B,C, and D, these are the keys the system will recognize as providing user input. Thus, a user must use these keys to interact with the program modules.

STIM_DURATION

The STIM_DURATION switch specifies the presentation time for a particular stimulus in milliseconds, for example. It determines how long the stimulus will be displayed on the display device. For example, some of the neuropsychological tests feature a presentation of stimuli to a test subject. A flashing snowflake, for example, may be presented on a display device as part of the particular neuropsychological test (for example, the Simple Reaction Test). During program execution, the NTP can select an option to specify the period and/or frequency upon which the star will flash (for example, five milliseconds) as well as the flash duration.

HAND

The HAND switch indicates which one of the test subject's hands is the response hand. For example, a value of ‘1’ may indicate the subject is left-handed, and a value of ‘2’ may indicate the subject is right-handed.

RESPONSE_DEVICE

The RESPONSE_DEVICE switch indicates the device the test subject is using to interact with the program modules. For example, a value of ‘0’ indicates that the subject is using a keyboard, and a value of ‘1’ indicates that the subject is using a mouse.

EYE

The EYE switch indicates which eye of the test subject is receiving the stimulus. For example, ‘0’ may indicate the right eye, and ‘1’ may indicate the left eye.

PRACTICE_MODE

The PRACTICE_MODE switch indicates the various levels of feedback for the practice modes. For example, a value of ‘0’ indicates that the practice mode is off, a value of ‘1’ indicates positive, a value of ‘2’ indicates negative, and a value of ‘3’ indicates both.

STIMULUS_SET

The STIMULUS_SET switch defines a stimulus set to be used for those tasks of a program module which are driven by external files (for example, memory search or mathematical processing). It requires that the filename which contains the stimulus set (more than one stimulus presentation) be specified.

RANDOM_SEED

The RANDOM_SEED switch seeds the random number generator. For example, a stimulus for a particular test may be presented in a random, preferably pseudo-random fashion. To generate the “randomness,” a seed can be specified to increase or decrease “randomness.”

TRIAL_TIMELIMIT

The TRIAL_TIMELIMIT indicates the length of time for a given trial. For example, a value less than zero indicates an “infinite” time (no timeout).

SOUND_TOGGLE

The SOUND_TOGGLE switch specifies whether a particular program module should use audio.

HEMISPHERE_ORDER

The HEMISPHERE_ORDER switch defines the brain hemisphere ordering for tasks that require it. For example, the switch may have a value of ‘R’ (indicating right brain), ‘L’ (indicating left brain), RL (indicating right brain/left brain), or LR (indicating left brain/right brain).

SUB_BLOCKS

The SUB_BLOCKS switch is used in conjunction with a task that includes definable sections. The switch allows definition of the order in which the sub-blocks are run. For example, 1341, may be used to define the block order for a particular task as 1, 3, 4, 1.

ISI_LEVEL

The ISI_LEVEL switch selects the predefined interstimulus level. For example, the value for the switch may be an integer representing a range of values (seconds, for example) that are randomly chosen.

SET_SIZE

The SET_SIZE switch defines the size of a set used by a particular program module. For example, the memory set size used by the Memory Search Test may be specified as four items per set.

NUMBER_OF_TRAILS

The NUMBER_OF_TRIALS switch specifies the number of stimuli presented by a particular task.

TARGET_SPEED

The TARGET_SPEED switch controls the speed of a moving cursor, for example. For example, the value of the switch may be specified in millimeters/second.

VIEW_DELAY

The VIEW_DELAY switch specifies the minimum view delay in milliseconds, for example, for a stimulus memory set.

In addition to the parameters described above, the present invention offers the NTP an option to alter a switch or parameter value for the contrast of a given stimulus, for example. Such features allow the NTP to tailor the program to meet the needs of specific individuals and to test for different conditions. For example, many elderly test subjects may have slower reflexes than their younger counterparts. Thus, their reaction time to the testing stimulus may be less. Accordingly, when taking the Simple Reaction Time Test, for example, elderly test subjects may require that the flashing snowflake not flash as it would for a younger subject. As a result, they may require that the flashing snowflake not flash as frequently as it would for a younger subject. However, the NTP may be interested in monitoring degradation of reaction time as a function of age and thus may not choose to alter the stimulus frequency.

E. Battery of Tests

In at least one embodiment of the present invention, a method in a computer system for administering at least one neuropsychological test battery is provided. The method includes the steps of displaying a menu including names of neuropsychological test batteries, receiving a selection relating to a neuropsychological test battery to be executed, allowing control of at least one parameter relating to a neuropsychological test in the test battery to be executed, and executing the neuropsychological test battery in accordance with the at least one parameter.

In at least one embodiment of the present invention, an article of manufacture is provided including a computer usable medium having computer readable program code means embodied therein for causing a computer to execute a test battery (a specific combination of individual neuropsychological testing program modules). The computer readable program code means in the article of manufacture also includes computer readable program code means for causing the computer to allow the NTP to control at least one parameter relating to a neuropsychological assessment test in the test battery.

A battery of tests for a specific condition can be chosen by the NTP. For example, in embodiments of the present invention, the NTP is presented with a menu with choices for execution of a neuropsychological test battery. Examples of the particular test batteries include the NeuroCognitive test battery which includes, for example, a Stanford Sleep Scale test, a Moodscale 2 test, a Simple Reaction Time test, a Spatial Processing test, a Code Substitution Test, a Matching to Sample test, a Mathematical Processing test, a Running Memory 5-Minute test, a Digital Set Comparison test, a Logical Reason test, a Code Substitution test, a Memory Search test, a Finger Tapping test, a Tower Puzzle test, and a Stroop test; the Moderate Cognitive test battery which includes a Sleep Scale test, a Mood Scale test, a Simple Reaction Time test, a 2-Choice Reaction Time test, a Code Substitution test, a Matching to Sample test, a Logical Relations test, a Delayed Memory test, a Sternberg-6 test, and a Sleep Scale test; a Sports Medicine test battery, an Attention Deficit Hypertension Disorder test battery, a Parkinson's Disease test battery, and a Pre-deployment test battery (for military personnel).

Just as each individual test may be introduced with an introductory screen (for example, a title screen), each test battery may be introduced with an introductory screen or a series of introductory screens. In some embodiments of the present invention, the NTP may specify information regarding where the test results of a battery will be stored. The menu program module also prompts the NTP for information relating to test subject identification, type of test, whether instructions are to be provided, instruction file extension, run number, dominant hand preference, and type of a run.

These variables operate in the same manner described in the library subsection. Similarly, subject information and demographics information may also be collected for the battery to be executed. In at least one embodiment of the present invention, the menu program module also prompts the user for configuration information. For example, configuration information preferably includes but is not limited to a specification indicating a particular drive to which data may be written (for example, a report of test results may be stored on an attachable disk drive or a supplementary disk drive), a file from which introductory information can be read (for example, a file containing user instructions for a particular test), and capability to modify content of an introductory screen, specification to specify alternate menulist files. These specifications can be stored in variables or data fields. Examples of these variables are as follows:

D_SWITCH_VARIABLE

The D Switch variable specifies a particular drive (for example, a diskette drive) to write data from a battery of tests that will be executed.

I SWITCH Variable

The I Switch variable allows the NTP to instruct the menu program module to read introductory information from a particular file (for example, a text file such as an ASCII file).

T SWITCH Variable

The T Switch variable allows the NTP to modify the content of the introductory screen.

In addition to offering the user a choice of neuropsychological assessment test batteries, the menu program module preferably presents the user with a screen including general information such as the title, sponsors, version revision, authors, and creators, as described above in the library subsection. As described above with respect to individual neurological assessment tests, before a test battery is executed, the menu program module preferably issues a prompt for information relating to test subject identification, a type of test, whether instructions are to be provided for a test, file extension for instruction files, a run number, a dominant hand preference, a paused or continuous indication (for indicating whether a resting period is allowed between tests), a type of run indication, audio sounds to be used during execution, and a specification for true randomization or pseudo randomization. Again, this information is preferably stored in variables or data fields. It should be noted that the above described list is meant to serve as an example. By no means, should the list be considered exhaustive. After being presented with the disclosure herein, those skilled in the relevant art(s) will appreciate that other information can be solicited as well. As described earlier, after this information has been input, the information may be changed as necessary.

In at least one embodiment of the present invention, the menu program module propagates the user responses to the variables (for example, test subject ID, type of test, etcetera) to each test within the selected test battery. For example, after a test battery for Parkinson's Disease is selected and the user is prompted for his responses to the above referenced variables, the menu program module transmits these variables to each individual neuropsychological assessment test within the test battery for Parkinson's Disease.

For example, the run number variable is transmitted to each individual neuropsychological assessment test within the battery to supply a seed for the stimulus random number generator, as the stimulus for each individual assessment test within the battery is pseudo-randomly seeded. For example, a run number of “2” will produce the same sequence of stimuli, but a different run number (for example, a run number of “3”) will produce a sequence that is different from run number 2 (although the same sequence of run number 3 will be repeated if run number 3 is selected). In other words, a given run number produces the same sequence of randomly generated stimuli, but the sequence for a given run number may, and preferably does differ from the sequence of another run number.

Similarly, the menu program module preferably transmits a variable indicating whether a test subject desires user test instructions to each individual neuropsychological test within a selected test battery to be executed. It should be noted, however, that these settings may be overwritten for a specific assessment test within the battery by respecifying the value of a variable at the beginning of the specific assessment test. For example, a user may have indicated that he desires user test instructions to be displayed for a test battery via the menu program module. If, however, a specific assessment test is to be repeated within the battery, the test subject may not need to view instructions for this particular test on subsequent executions of the test.

Thus, the test subject may indicate that user instructions should not be provided for the particular test, thereby overriding the instruction variable for this particular test. The present invention preferably accomplishes this by providing the user with an option to display instructions prior to beginning a specific test within a test battery. It should also be noted, that in at least one embodiment of the present invention, the test subject is provided with an option through which he can specify that the menu program module read a text file including information related to each individual neuropsychological assessment test within a given test battery. In such a situation, user-controllable parameters specific to each individual test can be specified directly from the text file (for example, an ASCII data file).

Now referring to FIG. 8, a diagram is shown in which three test batteries A, B, and C are shown. Each of the test batteries A, B, and C represent three distinct test batteries for assessing three different conditions. For example, test battery A includes individual neuropsychological tests 1, 3, and 9 and may be employed to assess Dementia, for example. Test battery B includes individual neuropsychological tests 2, 6, and 4 and may be employed to assess Alzheimer's, for example, and test battery C includes neuropsychological tests 5, 3, and 6 and may be employed to assess Dyslexia, for example. It should be noted that although each battery assesses a different neuropsychological condition, individual batteries may have one or more individual neuropsychological tests in common. For example, test battery A and test battery C of FIG. 4 both include individual neuropsychological assessment test 3. It should also be noted that the number of individual neuropsychological assessment tests in each battery might vary. For example, test battery A could have more than three tests or any lesser number of tests, as could any of the other test batteries.

Upon choosing the menu selection of Parkinson's Disease, for example, the present invention allows a user to execute a neuropsychological assessment test battery for Parkinson Disease which includes a subset of individual neuropsychological assessment tests from the library of tests described above. This subset includes individual neuropsychological tests that have preferably been determined by neuropyschology professionals to be effective in testing for Parkinson's Disease. Before the test battery is executed, however, in some embodiments, the test subject is offered an opportunity to alter certain user-controllable parameters or switches relating to the individual tests within the test battery. As mentioned above, the user-controllable parameter feature is sometimes more desirable than hard coding the parameters, as a change to hard-coded parameters requires intervention by a computer professional such as a computer instructional code programmer. However, for purposes of standardization, it may be desirable to hard code parameters in certain applications.

F. Battery Builder

In accordance with a particularly advantageous aspect of the invention, the NTP or the user may create its own battery of tests to test for a specific condition. For example, in one embodiment, upon receiving a menu selection indicating that an NTP desires to create a tailor-made battery, the present invention provides the NTP with a choice including all of the individual neuropsychological assessment tests included within the library described above. The NTP may then select the individual neuropsychological assessment tests that comprise the battery from amongst the library of neuropsychological assessment tests of the present invention. It should be noted that the NTP may select any number of assessment tests from a single test to the entire library. This aspect of the invention is useful for assessing conditions for which batteries have not yet been constructed. It is also useful for conducting research, as the NTP can experiment with various combinations of individual neuropsychological assessment tests without being confined to predefined batteries.

Now referring to FIG. 9, a flow diagram illustrating the steps involved in creating a customized test battery is shown. Control begins with step 505 and proceeds immediately to step 910.

In step 910, the NTP selects a specific combination of neuropsychological assessment tests from the plurality of individual neuropsychological assessment tests within the library. For example, the NTP may build a specific battery for assessing a specific condition by selecting individual neuropsychological assessment tests that correspond to the specific condition. For example, the creator of a specific test battery may select the individual neuropsychological tests 1, 3, and 9 from the library of individual neuropsychological assessment tests. The resulting combination of individual neuropsychological assessment tests comprise a customized test battery useful for assessing test subjects for a particular neuropsychological condition. After selecting the individual tests to be included in the battery, the user is preferably given the option of changing the user-controllable parameters relating to the various selected assessment tests. In at least one embodiment of the present invention, before being allowed to select the individual neuropsychological tests to be included in the battery, the user is presented with an option by which he can enter a specific name for the battery he will create for identification purposes. In such an embodiment, the user preferably is allowed to save the battery for future testing.

In some situations, the NTP may desire to repeat a specific test(s) in a battery. The present invention allows the NTP to specify that a test or tests within a test battery is to be repeated. Thus, in decision step 915, the NTP is asked whether any tests should be repeated. If it is determined that the NTP desires to repeat at least one test, control resumes with step 920. In step 920, the NTP is prompted to enter or select the name of the individual neuropsychological test(s) he desires to have repeated. For example, the NTP may elect to have test 3 of test battery A (FIG. 8) repeated a specified number of times.

In decision step 925, it is determined whether the NTP desires to have certain conditions met before a test(s) is repeated. If it is determined that certain conditions must be met before a test(s) is repeated, control resumes with step 930.

In step 930, the NTP enters or selects the conditions that must occur for an individual test(s) within the test battery to be repeated. For example, the NTP may desire to have individual neuropsychological assessment test 3 of test battery A (FIG. 4) repeated twice only if the test subject achieves a certain score (for example, only if the test subject responds incorrectly to three out of five stimuli presentations) in the test. In such a situation, in step 930, the NTP would enter this as a condition or select this as a condition from a group of condition choices.

Returning to decision step 915, if it is determined that the NTP does not wish to repeat any tests, control resumes with step 935. Similarly, if in decision step 925, it is determined that there are no special conditions for repeating a test, control resumes with step 935.

In step 935, the NTP selects the order that each of the individual neuropsychological assessment tests are to be administered. For example, individual neuropsychological assessment test 3 may be administered first, followed by individual neuropsychological assessment test 9, followed by individual neuropsychological assessment test 1 in test battery A of FIG. 8 for example. Finally, control ends with step 940.

### VII. CONCLUSION

Those skilled in the art will appreciate that various adaptations and modifications of the above-described embodiments can be configured without departing from the scope and spirit of the present invention. Therefore, it is to be understood that, within the scope of the appended claims, the invention may be practiced and constructed other than as specifically described herein.

1. An automated neuropsychological testing system, comprising:
a plurality of computer program test modules, each program module including computer readable instructions for administering a neuropsychological assessment test;
a computer program control module which allows a user to control at least one parameter of said neuropsychological assessment test;
a microprocessor for executing said computer program test modules and said computer program control module to administer the neuropsychological assessment test;
an input device in communication with said microprocessor for allowing said user to interact with said computer program test modules and computer program control module; and
an output device in communication with said microprocessor, said output device operable to provide information relating to each neuropsychological assessment test.

2. The system of claim 1 further comprising a statistical program module for calculating statistics from results of the administration of the neuropsychological assessment test.

3. The system of claim 2, wherein said statistics include at least one of mean, median, reaction time, accuracy, and throughput.

4. The system of claim 1, wherein said output device includes a display that displays textual information and each program module includes computer readable instructions for adjusting font of textual information.

5. The system of claim 1 wherein the at least one parameter includes at least one of a type of test, a dominant hand, a type of test run, a mode of test run, an indication of whether to display test results, a feedback mode, a random number seed, a response device and an indication of whether to show results of a test battery.

6. A method in a computer system for administering a neuropsychological test comprising:
displaying a menu including plurality neuropsychological assessment tests;
receiving a selection of a neuropsychological test for execution;
configuring at least one parameter relating to the selected neuropsychological test responsive to user input; and
executing the selected neuropsychological test.

7. The method of claim 6 wherein executing the test includes:
displaying test instruction responsive to a request for instructions;
displaying stimuli; and
accepting test subject's response to the stimuli.

8. A method in a computer system for administering a neuropsychological test battery comprising:
displaying a menu including plurality neuropsychological assessment test batteries;
receiving a selection of a neuropsychological test battery for execution;
configuring at least one parameter relating to the selected neuropsychological test battery responsive to user input; and
executing the selected neuropsychological test battery.

9. A method in a computer system of building a neuropsychological test battery comprising:
selecting one or more neuropsychological tests from among a plurality of neuropsychological tests;
producing a query as to whether any of the selected tests are to be repeated;
producing a query as to requesting conditions that must occur in order for the selected tests to be repeated; and
selecting an order in which the selected tests are to be administered.

10. The method of claim 9 further comprising receiving a response indicating the neuropsychological test to be repeated.

11. The method of claim 11 further comprising a receiving a response indicating a number of repetitions for the neuropsychological test to be repeated.

12. The method of claim 11 further comprising receiving conditions that must occur in order for the selected test to be repeated.

13. The method of claim 9 further comprising configuring at least one parameter for at least one of the selected neuropsychological tests responsive to user input.

14. The method of claim 13 wherein the at least one parameter includes a switch indicating a storage location to which data from the neuropsychological battery will be stored.

15. The method of claim 13 wherein the at least one parameter includes a switch indicating a duration of presentation of a stimulus associated with one or more the neuropsychological tests.

16. The method of claim 13 wherein at the at least one parameter includes a switch indicating a test subject's response hand.

17. The method of claim 13 wherein at the at least one parameter includes a switch indicating which eye of the test subject receives stimulus from the one or more neuropsychological tests.

18. The method of claim 13 wherein at the at least one parameter includes a switch identifying a device that a test subject is using to respond to stimuli.

19. The method of claim 18 wherein the device identified by the switch is a keyboard.

20. The method of claim 18 wherein the device identified by the switch is a mouse.
